(19) United States
(12) Patent Application Publication
(10) Pub. No.: US 2004/0121971 A1
(43) Pub. Date: Jun. 24, 2004

(54) Therapeutic use of tumor necrosis factor-alpha mutein

(76) Inventors: Gang Chen, Shanghai (CN); Xinjiang Du, Shanghai (CN); Dezheng Wu, Beijing (CN); Weijing Zhang, Beijing (CN)

Correspondence Address: WILSON SONSINI GOODRICH & ROSATI, 650 PAGE MILL ROAD, PALO ALTO, CA, 943041050

(21) Appl. No.: 10/327,619

(22) Filed: Dec. 20, 2002

(51) Int. Cl. A61K 48/00; A61K 38/19

(52) U.S. Cl. 514/44; 424/85.1

(57) ABSTRACT

Improved methods for treating neoplastic diseases such as cancer are provided by using muteins of human tumor necrosis factor-alpha (TNF-α). Compared to wild-type human TNF-α these therapeutic TNF muteins have higher specific anti-tumor activity, but with much reduced systemic toxicity and milder side effects such chills and fever. In addition, potentially synergistic, novel combinations of the inventive TNF-α muteins with other anti-neoplastic agents are provided for effectively treating patients having particular types of cancer or malignancy or at particular stages of cancer development.

### BACKGROUND OF THE INVENTION

1. Field of the Invention

This invention relates to compositions and methods for using recombinant human tumor necrosis factor-alpha (TNF-α) muteins for therapeutic purposes, such as various forms of cancer, hematological disorders and diseases associated with abnormal angiogenesis, and further relates to combination therapy of the TNF-α muteins with other anti-neoplastic agents.

2. Description of Related Art

The evolution of new therapies for diseases associated with abnormal cell proliferation such as cancer has provided many choices of therapeutics for clinical treatment. Recent development and FDA approval of biologic therapy for refractory tumors, such as melanoma, raises a new hope that advanced tumors that have been refractory to all approaches with conventional drugs may be curable by using novel protein therapeutics with minimum side effects compared to the conventional chemotherapy.

1. Clinical Cancer Therapy

Currently therapeutic agents used in clinical cancer therapy are categorized into six groups: alkylating agents, antibiotic agents, antimetabolic agents, biologic agents, hormonal agents, and plant-derived agents.

The alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. The alkylating agents are cell cycle phase-nonspecific agents because they exert their activity independently of the specific phase of the cell cycle. The nitrogen mustards and alkyl alkone sulfonates are most effective against cells in the G₁ or M phase. Nitrosoureas, nitrogen mustards, and aziridines impair progression from the G₁ and S phases to the M phases. Chabner and Collins eds. (1990) “Cancer Chemotherapy: Principles and Practice”, Philadelphia: J B Lippincott.

The alkylating agents are active against wide variety of neoplastic diseases, with significant activity in the treatment of leukemias and lymphomas as well as solid tumors. Clinically this group of drugs is routinely used in the treatment of acute and chronic leukemias; Hodgkin's disease; non-Hodgkin's lymphoma; multiple myeloma; primary brain tumors; carcinomas of the breast, ovaries, testes, lungs, bladder, cervix, head and neck, and malignant melanoma. The major toxicity common to all of the alkylating agents is myelosuppression. Gastrointestinal adverse effects of variable severity occur commonly and various organ toxicities are associated with specific compounds. Black and Livingston (1990) Drugs 39:489-501; and 39:652-673.

The antibiotic agents are a group of drugs that produced in a manner similar to antibiotics as a modification of natural products. Examples of antibiotic agents include anthracyclines (e.g. doxorubicin, daunorubicin, epirubicin, idarubicin and anthracenedione), mitomycin C, bleomycin, dactinomycin, plicatomycin. These antibiotic agents interferes with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death.

The antibiotic agents have been used as therapeutics across a range of neoplastic diseases, including carcinomas of the breast, lung, stomach and thyroids, lymphomas, myelogenous leukemias, myelomas, and sarcomas. The primary toxicity of the anthracyclines within this group is myelosuppression, especially granulocytopenia. Mucositis often accompanies the granulocytopenia and the severity correlates with the degree of myclosuppression. There is also significant cardia toxicity associated with high dosage administration of the anthracyclines.

The antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine.

Antimetabolic agents have widely used to treat several common forms of cancer including carcinomas of colon, rectum, breast, liver, stomach and pancreas, malignant melanoma, acute and chronic leukemia and hair cell leukemia. Many of the adverse effects of atnimetabolite treatment result from suppression of cellular proliferation in mitotically active tissues, such as the bone marrow or gastrointestinal mucosa. Patients treated with these agents commonly experience bone marrow suppression, stomatitis, diarrhea, and hair loss. Chen and Grem (1992) Curr. Opin. Oncol. 4:1089-1098.

The hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Hormonal agents are used to treat breast cancer, prostate cancer, melanoma and meningioma. Because the major action of hormones is mediated through steroid receptors, 60% receptor-positive breast cancer responded to first-line hormonal therapy; and less than 10% of receptor-negative tumors responded. The main side effect associated with hormonal agents is flare. The frequent manifestations are an abrupt increase of bony pain, erythema around skin lesions, and induced hypercalcemia.

Plant-derived agents are a group of drugs that are derived from plants or modified based on the molecular structure of the agents. Examples of plant-derived agents include vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission.

Plant-derived agents are used to treat many forms of cancer. For example, vincristine is used in the treatment of the leukemias, Hodgkin's and non-Hodgkin's lymphoma, and the childhood tumors neuroblastoma, rhabdomyosarcoma, and Wilms' tumor. Vinblastine is used against the lymphomas, testicular cancer, renal cell carcinoma, mycosis fungoides, and Koposi's sarcoma. Doxetaxel has shown promising activity against advanced breast cancer, non-small cell lung cancer (NSCLC), and ovarian cancer. Etoposide is active against a wide range of neoplasms, of which small cell lung cancer, testicular cancer, and NSCLC are most responsive.

The plant-derived agents cause significant side effects on patients being treated. The vinca alkaloids display different spectrum of clinical toxicity. Side effects of vinca alkaloids include neurotoxicity, altered platelet function, myelosuppression, and leukopenia. Paclitaxel causes dose-limiting neutropenia with relative sparing of the other hematopoietic cell lines. The major toxicity of the epipophyllotoxins is hematologic (neutropenia and thrombocytopenia). Other side effects include transient hepatic enzyme abnormalities, alopenia, allergic reactions, and peripheral neuropathy.

Biologic agents are a group of biomolecules that elicit cancer/tumor regression when used alone or in combination with chemotherapy and/or radiotherapy. Examples of biologic agents include immuno-modulating proteins such as cytokines, monoclonal antibodies against tumor antigens, tumor suppressor genes, and cancer vaccines.

Cytokines possess profound immunomodulatory activity. Some cytokines such as interleukin-2 (IL-2, aldesleukin) and interferon-α (IFN-α) demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself.

Interferon-α includes more than 23 related subtypes with overlapping activities. IFN-α has demonstrated activity against many solid and hematologic malignancies, the later appearing to be particularly sensitive. Examples of interferons include, interferon-α, interferon-β (fibroblast interferon) and interferon-γ (fibroblast interferon). Examples of other cytokines include erythropoietin (epoietin-α), granulocyte-CSF (filgrastin), and granulocyte, macrophage-CSF (sargramostim). Other immuno-modulating agents other than cytokines include bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein.

Another example of monoclonal antibodies against tumor antigens is RITUXAN® (Rituximab) that is raised against CD20 on lymphoma cells and selectively deplete normal and maligant CD20⁺ pre-B and mature B cells. RITUXAN® is used as single agent for the treatment of patients with relapsed or refractory low-grade or follicular, CD20⁺, B cell non-Hodgkin's lymphoma.

MYELOTARG® and CAMPATH® are further examples of monoclonal antibodies against tumor antigens that may be used.

Tumor suppressor genes are genes that function to inhibit the cell growth and division cycles, thus preventing the development of neoplasia. Mutions in tumor suppressor genes cause the cell to ignore one or more of the components of the network of inhibitory signals, overcoming the cell cycle check points and resulting in a higher rate of controlled cell growth—-cancer. Examples of the tumor suppressor genes include DPC-4, NF-1, NF-2, RB, p53, WT1, BRCA1 and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAAs include gangliosides (GM2), prostate specific antigen (PSA), α-fetoprotein (AFP), carcinoembryonic antigen (CEA) (produced by colon cancers and other adenocarcinomas, e.g. breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

2. Tumor Necrosis Factor-α

Tumor necrosis factor-α (TNF-α), also known as cachectin, is a multifunctional cytokine produced mainly by activated macrophages. In vitro, it has diverse biological effects (reviewed by Manogue and Cerami (1988) in “Cellular and Molecular Aspects of Inflammation), Eds. Poste and Crooke, Plenum Press, New York, pp. 123-150; Chen and Goeddel (2002) Science 296:1634-1635; and Bodmer et al. (2002) Trends Biochem. Sci. 27:19-26), including killing of transformed cells (Carswell et al., (1975) Proc. Natl. Acad. Sci. USA 72:3666-3670), stimulation of granulocytes and fibroblasts (Old (1985) Science 230:630-632); Vilchek et al. (1986) J. Exp. Med. 163:632-643); Beutler and Cerami (1987) Nature 316:552-554), damage to endothelial cells (Sato et al, (1986) J. Natl. Cancer Inst. 76:1113), and anti-parasitic effects (Taveme et al. (1984) Clin. Exp. Immunol. 67:1-4). In vivo, it plays a key role as an endogenous mediator of inflammatory, immune and host defense functions and it is involved in a number of pathological conditions in man and mouse such as septic shock, cachexia, capillary leak syndrome, hemorhagic necrosis of multiple organs, etc. It is capable of acting independently and in conjunction with other factors affecting a whole plethora of different body functions. These effects can either be beneficial or life-threatening to the host. Some of these effects are direct, others may be mediated via the induction of other secreted factors. The biological effects of TNF-α are mediated via binding to specific cell surface receptors.

Members of the TNF superfamily play pivotal roles in the organization and function of the immune system. They share a common structural motif, the TNF homology domain (THD), which binds to cysteine-rich domains (CRDs) of TNF receptors. Bodmer et al. (2002) Trends Biochem. Sci. 27:19-26. The primary structures of human and mouse TNF-α (Pennica et al. (1984) Nature 312:724-729); and Fransen et al. (1985) Nucl. Acid. Res. 13:4417-4429) and of two different TNF-receptors (p55-TNF-R and p75-TNF-R) have been deduced from the nucleotide sequence of the cloned cDNA. Both receptors bind not only TNF-α but also TNF-β or lymphotoxin with high affinity (Schoenfeld et al. (1991) J. Biol. Chem. 266:3863-3869). TNF-β is a related lymphocyte product that exhibits pleiotropic activities very similar to those of TNF-α. TNF-α and TNF-β share 32% homology on the amino acid sequence level. X-ray crystallographic analysis revealed that the tertiary structure of both molecules is virtually identical except that the TNF-α trimer creates a molecule that is less elongated than the TNF-β trimer and the latter has a top-region that flares open (Eck et al. (1992) J. Biol. Chem. 267:2119-2122).

Besides the cell receptor interaction, TNF-α has been shown to have a lectin-like property for the oligosaccharide ligands chitobiose and Man (α3)-(Man(α1,6)-Man (Hession et al. (1987) Science 237:1479-1484); and Sherblom et al. (1988) J. Biol. Chem. 263:5418-5424). They further demonstrated that the TNF-α protein has at least two different binding sites, one lectin-like and the other directed at cell surface receptors.

Several TNF-α muteins were described for which the binding to the cellular TNF-receptor p55 and/or p75 was hampered. All of the mutations are located in the lower half of the pyramidal structure of the biologically active TNF-α trimer (Van Ostade et al. (1991) EMBO J. 10:827-836; and Van Ostade et al. (1992) Nature 361:266-269; and EP-A-0 486 908).

TNF-α has been implicated to be the principle mediator of endotoxic or septic shock (Cerami and Beutler (1988) Immunol. Today 9:28-31). Septic shock develops in the presence of severe infection, especially following bacteremia with Gram-negative bacteria and release of endotoxin. It may also be caused by any class of microorganism, including Gram-positive bacteria, viruses, fungi, protozoa, spirochetes and rickettsiae. Death is caused by progression to multiple organ failure and circulatory collapse. It has been found that the intravenous injection of LPS (or endotoxin) in animals produced several parameters of the septic shock syndrome, namely, hypotension, decreased systemic vascular resistance, leukopenia, thrombocytopenia, and tissue dammage. In experimental animals, TNF-α produces hypotension, leukopenia, and local tissue necrosis (Okusawa et al. (1988) J. Clin. Invest. 81:1162-1172). Administration of anti-TNF-α antibodies to baboons (Tracey et al. (1987) Nature 330:662-664) or rabbits prevents the shock induced by endotoxin (Mathison et al. (1988) J. Clin. Invest. 81:1925-1937).

In addition to its immunomodulating activity, TNF-α has also been shown to be involved in the control of growth and differentiation of various parasites. Upon infection of the host, parasites are capable of inducing the secretion of different cytokines such as TNF which may affect the course of the disease. For instance, in the case of malaria, TNF-α can be protective in certain circumstances, such as inhibiting parasite survival in rodent malaria (Clark et al. (1987) J. Immunol. 139:3493-3496; and Taverne et al. (1987) Clin. Exp. Immunol. 67:1-4). By contrast, its overproduction can be detrimental to the host and can contribute to the pathology of the disease (Clark (1987), supra; and Grau et al. (1989) Res. Immunol. 155:355-363).

The availability of recombinant TNF has enhanced its use in tumor therapy. However, the in vivo tumoricidal effects of TNF-α have always been accompanied by toxic side effects such as hypotension, abnormal liver function, leukopenia, chill and thrombus formation. Different approaches were followed to overcome these noxious effects, including the use of monoclonal antibodies as well as fragments thereof, which neutralize the in vitro and in vivo toxic properties of TNF-α (e.g., EP-A-0 350 690).

As described above, although thousands of potential anticancer agents have been evaluated, the treatment of human cancer remains fraught with complications and side effects which often present an array of suboptimal treatment choices. Despite the great number of anti-neoplastic agents that are used in the clinic for cancer treatment, a need still exists for drug regimens with higher therapeutic indexes for treating neoplastic diseases such as cancer.

### SUMMARY OF THE INVENTION

The present invention provides new and improved compositions, kits, and methods for treating neoplastic diseases such as cancer. In particular, muteins of human TNF-α are provided as therapeutic proteins that circumvent the problems associated with wild-type TNF-α such as systemic toxicity and various other side effects. In addition, potentially synergistic, novel combinations of the inventive TNF-α muteins with other anti-neoplastic agents are provided for treating patients having particular types of cancer or malignancy or at particular stages of cancer development.

In one aspect of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α [SEQ ID NO: 1] as having a deletion of amino acid residues at positions 1-7, wherein the amino acid residue positions are numbered relative to the N-terminus of hTNF-α.

According to the embodiment, the hTNF-α mutein further has a substitution of amino acid residue either at position 156 or 157 with a residue selected from the group consisting of Gln, Ser, Thr, Tyr, and Asn.

Also according to the embodiment, the hTNF-α mutein further has a substitution of one or more of amino acid residues at positions 8-10 with Lys or Arg.

Also according to the embodiment, the hTNF-α mutein has an amino acid sequence comprising SEQ ID NO: 2, 3, 4, or 5.

Also according to the embodiment, the hTNF-α mutein is encoded by a DNA sequence comprising SEQ ID NO: 10.

The disease associated with abnormal proliferation of cells can be a wide variety of indications or malignancy such as hematological disorders, cancer, malignant pleural effusion, malignant ascite, restenosis, and inflammatory diseases.

Hematologic disorders include abnormal growth of blood cells which can lead to dysplastic changes in blood cells and hematological malignancies such as various leukemias. Examples of hematological disorders include but are not limited to acute myeloid leukemia, acute promyelocytic leukemia, acute lymphoblastic leukemia, chronic myclogenous leukemia, the myelodysplastic syndromes, and sickle cell anemia.

Examples of cancers include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, mycloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

In a particular embodiment, the cancer is lung cancer, including small cell lung cancer (SCLC) and non-small cell lung cancer (NSCLC) such as squamous (epidermoid) carcinoma, adenocarcinoma (including bronchoalveolar), and large-cell (undifferentiated) carcinoma.

In another particular embodiment, the cancer is melanoma or skin cancer, preferably malignant or metastatic melanoma.

In yet another particular embodiment, the cancer is lymphoma including both Hodgkin's disease and non-Hodgkin's lymphomas, preferably malignant lymphoma, and more preferably non-Hodgkin's lymphomas.

In yet another particular embodiment, the disease associated with abnormal proliferation of cells is malignant pleural effusion (fluid in the chest cavity) that may be caused by cancer or other malignancy.

In yet another particular embodiment, the disease associated with abnormal proliferation of cells is malignant ascite (fluid in the abdominal cavity) that may be caused by cancer or other malignancy such as hepatitis.

In another embodiment of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α [SEQ ID NO: 1] as having a substitution of amino acid residues at positions 80, 90, and 92, wherein the amino acid residue positions are numbered relative to the N-terminus of hTNF-α.

According to the embodiment, the hTNF-α mutein has an amino acid sequence comprising SEQ ID NO: 6 or 7.

In yet another embodiment of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α [SEQ ID NO: 1] as having a substitution of amino acid residue at position 2, wherein the amino acid residue positions are numbered relative to the N-terminus of hTNF-α.

According to the embodiment, the hTNF-α mutein has an amino acid sequence comprising SEQ ID NO: 8 or 9.

The hTNF-α mutein may be delivered to the patient via various routes of administration. The mutein may be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The hTNF-α mutein may also be administered or coadministered in slow release dosage forms.

In a particular embodiment, the hTNF-α mutein is administered intravenously.

In another particular embodiment, the hTNF-α mutein is administered loco-regionally, such as intratumorally, intraperitoneally, by isolated limb perfusion, by isolated lung perfusion, by isolated liver perfusion, or by intravesical or intra-arterial infusion.

In a preferred embodiment, the hTNF-α mutein is administered into the patient via i.v. injection per day for 3-5 days per week at a dose preferably ranging from 0.1-100 μg/m², more preferably ranging from 0.5-50 μg/m², and most preferably from 1-20 μg/m². The preferred dosage below 100 μg/m² for the hTNF-α mutein is considered to be much lower than that used in the clinical trial of the recombinant wild-type hTNF-α (within a few mg/m² range). The preferred treatment cycle is 3-4 weeks.

Optionally, the hTNF-α mutein is administered into the patient via intravenous injection per day for 3-5 days per week at a dose preferably 100,000-1,000,000 unit/m², more preferably 200,000-800,000 unit/m², and most preferably 400,000-800,000 unit/m². The preferred treatment cycle is 3-4 weeks. The unit of the hTNF-α mutein is defined according the international standard as described in Meager and Das (1994) J. Immuno. Methods 170:1-13.

Also optionally, the hTNF-α mutein is administered into the patient via intraperitoneal injection per day for 1-2 days per week at a dose of preferably 500,000-5,000,000 units, more preferably 1,000,000-3,000,000 units, and most preferably of 2,000,000-3,000,000 units. The preferred treatment cycle is 2-3 weeks.

Alternatively, the hTNF-α mutein can also be expressed from an expression vector administered to a patient in a gene therapy-type of treatment. For example, viral vectors or plasmids encoding the hTNF-α mutein, such as recombinant retrovirus, adenovirus, adeno-associated virus, herpes simplex virus, vaccinia virus, and mammalian expression plasmids, may be employed to transduce or transfect cells of the patients.

### BRIEF DESCRIPTION OF THE FIGURES

FIG. 1 shows amino acid sequences of wild-type human TNF (157 aa, SEQ ID NO: 1) and its mutants (SEQ ID NOs: 2-9).

FIG. 2 shows the DNA sequence (SEQ ID NO: 10) encoding a rhTNFm1 (SEQ ID NO: 3) with methionine at the N-terminus.

FIG. 3 shows the amino acid sequence of the leader sequence of wild-type hTNF-α.

### DETAILED DESCRIPTION OF THE INVENTION

The present invention provides new and improved compositions, kits, and methods for treating neoplastic diseases such as cancer. In particular, muteins of human TNF-α are provided that contain deletions and substitutions in the N-terminus of the wild-type human TNF-α (h TNF-α), and optionally substitutions in the C-terminus. These mutations of TNF-α surprisingly render the TNF-α muteins much less toxic than the wild-type TNF-α and confer to the muteins high selectivity and specificity of types of cancer in clinical treatments of patients. These muteins have substantially reduced systemic toxicity and minimized side effects commonly associated with administration of the recombinant wild-type hTNF-α protein such as hypotension, abnormal liver function, leukopenia, chill and thrombus formation. Further, clinical data obtained by the inventors suggest that potentially synergistic, novel combinations of the inventive TNF-α muteins with other anti-neoplastic agents can be used to treat patients having particular types of cancer or malignancy (e.g., malignant pleural effusion and ascites) to significantly improve the response rate and to reduce patients' resistance to these anti-neoplastic agents. In addition, by using the compositions and methods of the present invention in the treatment, the patient's quality of life can be significantly improved with much less pain and suffering associated with the conventional chemotherapy.

1. TNF-α Muteins and Methods of Use

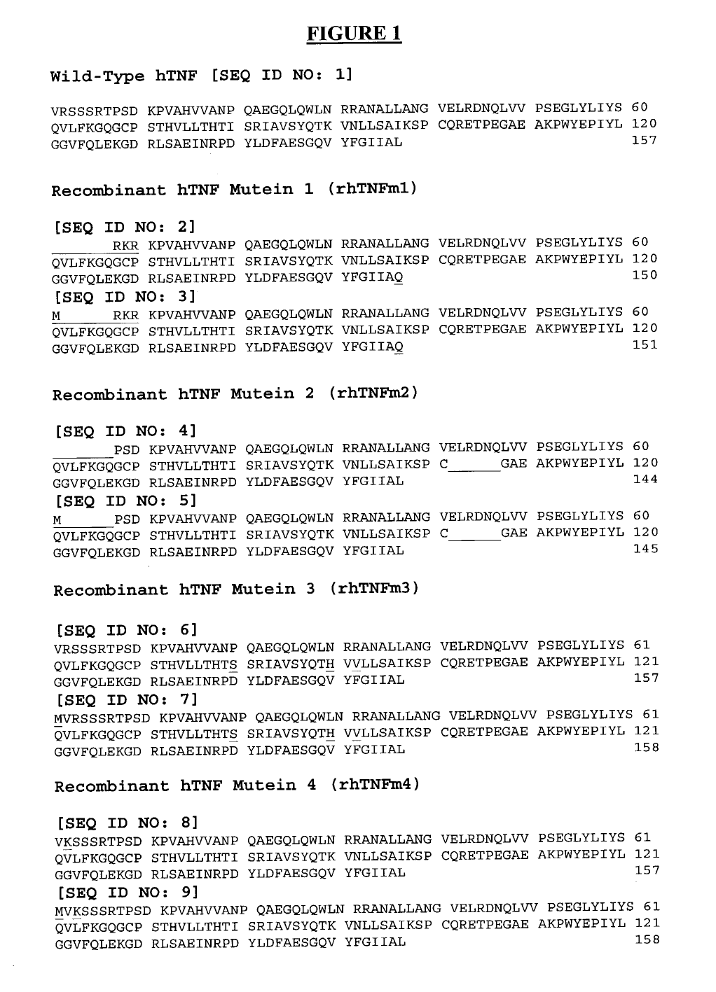

In one aspect of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α (SEQ ID NO: 1) (FIG. 1) as having a deletion of amino acid residues at positions 1-7, wherein the residue positions are numbered relative to the N-terminus of hTNF-α.

As shown in the Example section below, an embodiment of the hTNF-α mutein has been found to possess superior anti-cancer activities to and much lower systemic toxicities than those of the wild-type hTNF-α. While not wishing to be bound by the theory proposed herein, the inventors believe that deletion of the first 7 amino acid residues at the N-terminus may have contributed to the enhancement of the anti-cancer activities of the hTNF-α mutein.

According to the embodiment, the hTNF-α mutein further has a substitution of amino acid residue either at position 156 or 157 with a residue selected from the group consisting of Gln, Ser, Thr, Tyr, and Asn. While not wishing to be bound by the theory proposed herein, the inventors believe that substitution of either of the last 2 amino acid residues at the C-terminus may have contributed to the reduction in systemic toxicity of the hTNF-α mutein.

Also according to the embodiment, the hTNF-α mutein further has a substitution of one or more of amino acid residues at positions 8-10 with Lys or Arg. While not wishing to be bound by the theory proposed herein, the inventors believe that substitution of the amino acid residues at positions 8-10 with alkaline amino acids such as Lys and Arg may enhance the anti-cancer activities of the hTNF-α mutein.

Also according to the embodiment, the hTNF-α mutein has an amino acid sequence comprising SEQ ID NO: 2, 3, 4, or 5. As shown in FIG. 1, the amino acid sequence of hTNF-α mutein 1 (hTNFm1) or hTNF-α mutein 2 (hTNFm2) may or may not include a methionine residue at the N-terminus.

Also according to the embodiment, the hTNF-α mutein is encoded by a DNA sequence comprising SEQ ID NO: 10.

In another embodiment of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α (SEQ ID NO: 1) as having a substitution of amino acid residues at positions 80, 90, and 92, wherein the residue positions are numbered relative to the N-terminus of hTNF-α.

According to the embodiment, the hTNF-α mutein has an amino acid sequence comprising SEQ ID NO: 6 or 7. As shown in FIG. 1, the amino acid sequence of hTNF-α mutein 3 (hTNFm3) may or may not include a methionine residue at the N-terminus.

In yet another embodiment of the invention, a method is provided for treating a patient having a disease associated with abnormal proliferation of cells. In one embodiment, the method comprises: administering to the patient a mutein of human tumor necrosis factor-alpha (hTNF-α), the hTNF-α mutein being defined based on the amino acid sequence of hTNF-α (SEQ ID NO: 1) as having a substitution of amino acid residue at position 2, wherein the residue positions are numbered relative to the N-terminus of hTNF-α.

According to the embodiment, the hTNF-α mutein has an amino acid sequence comprising SEQ ID NO: 8 or 9. As shown in FIG. 1, the amino acid sequence of hTNF-α mutein 4 (hTNFm4) may or may not include a methionine residue at the N-terminus.

It is noted that the TNF-α muteins of the present invention include the prohormone and mature hormones forms. For the wild-type human TNF-α the prohormone is a 26 kD molecule, whereas mature hormone is a 17 kD molecule (157 aa, SEQ ID NO: 1) that results from the removal of a 76 amino acid leader sequence (SEQ ID NO: 11) (FIG. 3). The prohormone is known to be membrane bound and is not freely circulating, whereas the mature hormone is not membrane bound and is free to circulate. Optionally, for the prohormone forms the 76 amino acid leader sequence may be removed and replaced with another leader sequence that facilitates the secretion of the mature hormone. A number of leader sequences will perform this function, such as the gamma interferon leader sequence, as described by Gray et al. (1982) Nature 295:503.

The TNF-α muteins of the present invention may also be modified on the amino acid residues while the antitumor activity is essentially preserved and the side effects are further reduced. For example, less than 5 amino residues per TNF trimer may be modified with retention of the native cytolytic biological activity. The TNF-α muteins modified with a lipophilic moiety may be conveniently formulated with liposomes and delivered to the patients with reduced side effects.

The amino residues of the TNF-α muteins that may be modified include the N-terminal amino group or lysine amino residues of the TNF molecule. These amino residues become reactive and facilitate the attachment of other chemical groups, such as fatty acids, to the TNF structure. The attachment of fatty acids to the modified TNF enhances the hydrophobicity of the TNF, thereby facilitating the efficient and highly stable association of the TNF to liposomes.

For example, the modified amino residues of TNF-α muteins may be lysine amino residues or an N-terminal amino residue. The lysyl side chains can function as attachment sites for fatty acids to the TNF-α muteins. The amino residues can be modified to include fatty acids, preferably long chain fatty acids such as those fatty acids having a carbon chain length of between 8 and 14 carbon atoms. Such modified TNF-α muteins associate with a liposome with high binding efficiency, preferably more than 80% association rate.

The TNF-α muteins of the present invention can also be modified by conjugation with linear or branched polymer. It is believed that conjugation of the TNF mutiens with polymer would extend the in vivo circulating life of these biologically active materials and reduce immunogenicity/antigenicity as compared with unmodified versions.

For example, poly(ethylene glycol) (PEG) and similar water-soluble poly(alkylene oxides), as disclosed in U.S. Pat. No. 4,179,337, the disclosure of which is incorporated herein by reference, may be used to modify the TNF-α muteins. To conjugate poly(alkylene oxides) (PAO), one of the hydroxyl end-groups is converted into a reactive functional group. This process is frequently referred to as “activation” and the product is called an “activated poly(alkylene oxide)”. Other substantially non-antigenic polymers are similarly “activated” or functionalized.

The activated polymers are reacted with a therapeutic agent having nucleophilic functional groups that serve as attachment sites. One nucleophilic functional group commonly used as an attachment site is the epsilon-amino groups of lysines. Free carboxylic acid groups, suitably activated carbonyl groups, oxidized carbohydrate moieties and mercapto groups have also been used as attachment sites.

Optionally, umbrella-like branched polymers of PAO or PEG (U-β AO's or U-PEG's), as disclosed in U.S. Pat. No. 6,114,906, the disclosure of which is incorporated herein by reference, may be used to modify the TNF-α muteins reacting with biologically active nucleophiles on the proteins to form conjugates. The point of polymer attachment depends upon the functional group attached to the protein. For example, the functional group can be a succinimidyl succinate or carbonate and react with epsilon amino lysines. Alternatively, the functional group can be a carboxylic acid which is capable of reacting with hydroxyl groups found on biologically-active nucleophiles to form ester-linked prodrugs. The branched polymers can also be activated to link with any primary or secondary amino group, mercapto group, carboxylic acid group, reactive carbonyl group or the like found on proteins. Other groups are apparent to those of ordinary skill in the art.

The TNF-α muteins of the present invention can also be labeled with radio-isotopes and used in radiotherapy by targeting cells bearing TNF-α receptors, similar to radiolabeled antibodies used in radioimmunotherapy. Radioimmunotherapy is most effectively conducted by binding radioactive atoms to monoclonal antibodies having affinities and/or specificities for a particular type of target cell.

Similar to those used in radiolabeling monoclonal antibodies, the radiolabeled atoms which may be utilized in labeling the TNF-α muteins are also known in the art (Hunter et al. (1962) Nature 194:495; and Hnatowich et al. (1983) Science 220:613. Typically, the antibodies are labeled with a radioactive atom such as ¹³¹I, ⁹⁰Y, ²¹²Bi, ¹⁸⁶Re, ²²¹At, ^{99m}Tc and mixtures thereof. These radioisotopes may be used to label the TNF-α muteins as well. However, other radioisotopes may also be utilized as is known in the art.

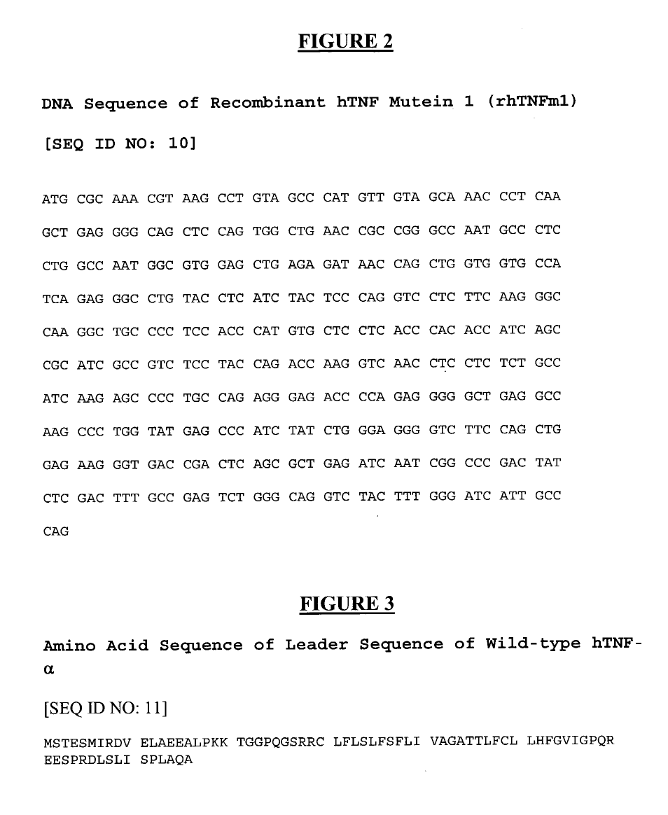

The TNF-α muteins of the present invention can also be expressed from an expression vector administered to a patient in a gene therapy-type of treatment. For example, viral vectors encoding the TNF-α muteins, such as recombinant retrovirus, adenovirus, adeno-associated virus, herpes simplex virus and vaccinia virus, may be employed to transduce cells of the patients. Alternatively, mammalian expression DNA plasmids (e.g., containing SEQ ID NO: 10 as shown in FIG. 2) encoding the TNF-α muteins may be administered as “naked DNAs” to the patients to transfect cells therein. These expression vectors may be engineered to express the membrane-bound TNF prohormones which are further processed in vivo to release soluble TNF-α muteins in the body.

The polynucleotides (SEQ ID NO: 10 as shown in FIG. 2) encoding the inventive TNF-α muteins can be cloned by recombinant techniques into vectors which are introduced to host cells where the fusion proteins can be expressed.

Generally, host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the polynucleotides encoding the TNF-α muteins. The culture conditions, such as temperature, pH and the like, are those previously used with the host cell selected for expression, and will be apparent to the ordinarily skilled artisan.

According to the invention, a recombinant vector is provided that comprises the polynucleotide sequence encoding a TNF-α mutein of the present invention. The recombinant vectors can be an expression vector for expressing the TNF-α mutein encoded by a nucleic acid in a host organism. The host organism includes, but is not limited to, mammalian (e.g., human, monkey, mouse, rabbit, etc.), fish, insect, plant, yeast, and bacterium.

Expression of the polynucleotide encoding the TNF-α muteins is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, a tetracycline or tetracycline-like inducible promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter for the wildtype TNF which controls the polynucleotide encoding the TNF-α muteins.

Also according to the invention, a recombinant cell is provided that is capable of expressing the polynucleotide sequence encoding the TNF-α muteins. The recombinant cell may constitutively or be induced in the presence or absence of an agent to express the TNF-α muteins in a host organism. The type of the recombinant cell includes, but is not limited to, mammalian (e.g., human, monkey, mouse, rabbit, etc.), fish, insect, plant, yeast, and bacterial cell.

2. Anti-Neoplastic Agents in Combination with TNF-α Mutein

Compositions according to the present invention might include a TNF-α mutein, a non-TNF-mutein agent, together with a pharmaceutical excipient. The composition preferably has a therapeutic synergy in the treatment of a disease, or a synergistic effect on the subject being treated. As used herein, a synergistic effect is achieved when a greater therapeutic effect results with a combination therapy than using either drug or monotherapy alone. One advantage of combination therapy with a synergistic effect is that lower dosages of one or both of the drugs or therapies may be used so that the therapeutic index is increased and toxic side effects are reduced.

A wide variety of anti-neoplastic agents may be used in conjunction with the combination of the TNF-α mutein of the present invention (e.g., rhTNFm1-4, FIG. 1) for treating various diseases associated with abnormal cell proliferation such as cancer. The particular anti-neoplastic agent(s) used in conjunction with the TNF-α mutein may depend on the particular type of cancer to be treated.

The antineoplastic agent may be an alkylating agent. The alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. A combination therapy of an alkylating agent and the TNF-α mutein of the present invention may have therapeutic synergistic effects on cancer and reduce sides affects associated with this chemotherapeutic agent.

The antineoplastic agent may be an antibiotic agent. Antibiotic agents are a group of anticancer drugs that are produced in a manner similar to antibiotics by a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and form an activated complex, which then binds to bases of DNA, causing strand scissions and cell death. A combination therapy of an antibiotic agent and the TNF-α mutein of the present invention may have therapeutic synergistic effects on cancer and reduce sides affects associated with this chemotherapeutic agent.

The antineoplastic agent may be an antimetabolic agent. Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. A combination therapy of an antimetabolic agent and the TNF-α mutein of the present invention may have therapeutic synergistic effects on cancer and reduce sides affects associated with this chemotherapeutic agent.

The antineoplastic agent may also be a plant-derived agent. Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), water soluble or insoluble camptothecin (e.g. 20(S)-camptothecin, 9-nitro-camptothecin, 9-amino-camptothecin, and topotecan), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Camptothecin is believed to be a potent inhibitor of the nuclear enzyme DNA topoisomerase I (topo-I), which is responsible for “relaxation” of supercoiled double-stranded DNA by creating single-stranded breaks through which another DNA strand can pass during transcription. Topo-I reseals the break allowing DNA replication to occur. Inhibition of topo-I leads to the formation of stable DNA-topoisomerase complexes, with eventual formation of irreversible double-stranded DNA breaks, leading to apoptosis and/or other forms of cell death. Podophyllotoxins such as etoposide are believed to interfere with DNA synthesis by interacting with topoisomerase II, leading to DNA strand scission. A combination therapy of a plant-derived agent and the TNF-α mutein of the present invention may have therapeutic synergistic effects on cancer and reduce side affects associated with these chemotherapeutic agents.

The antineoplastic agent may be a biologic agent. Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy of the biologic agent and the TNF-α mutein of the present invention may have therapeutic synergistic effects on cancer, enhance the patient's immune responses to tumorigenic signals, and reduce potential sides affects associated with this biologic agent.

Cytokines possess profound immunomodulatory activity. Some cytokines such as interleukin-2 (IL-2, aldesleukin) and interferon-α (IFN-α) demonstrate antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and non-self. Examples of interleukins that may be used in conjunction with the TNF-α mutein of the present invention include, but are not limited to, interleukin 2 (IL-2), and interleukin 4 (IL-4), interleukin 12 (IL-12).

Interferon-α includes more than 23 related subtypes with overlapping activities, all of the IFN-α subtypes within the scope of the present invention. IFN-α has demonstrated activity against many solid and hematologic malignancies, the later appearing to be particularly sensitive. Examples of interferons that may be used in conjunction with the TNF-α mutein of the present invention, but are not limited to, interferon-α, interferon-β (fibroblast interferon) and interferon-γ (fibroblast interferon).

Other cytokines that may be used in conjunction with the TNF-α mutein of the present invention include those cytokines that exert profound effects on hematopoiesis and immune functions. Examples of such cytokines include, but are not limited to erythropoietin (epoietin-α), granulocyte-CSF (filgrastin), and granulocyte, macrophage-CSF (sargramostim). These cytokines may be used in conjunction with the TNF-α mutein of the present invention to reduce chemotherapy-induced myelopoietic toxicity.

Immuno-modulating agents other than cytokines may also be used in conjunction with the TNF-α mutein of the present invention to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

The TNF-α mutein of the present invention may also be used in combination with other members of the TNF superfamily, including but not limited to, wild-type TNF-α, TNF-β, LT-β, OPGL, Fas ligand (FasL), TRAIL, CD27 ligand (CD27L), CD30 ligand (CD3OL), CD40 ligand (CD40L), 4-1BB ligand (4-1BBL), DcR3, OX40L, TNF-γ (WO 96/14328), AIM-I (WO 97/33899), endokine-oc (WO 98/07880), OPG, and neutrokine-a (WO 98/18921), OX40, and nerve growth factor (NGF).

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including the TNF-α mutein of the present invention and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and maligant CD20⁺ pre-B and mature B cells. RITUXAN® is used as single agent for the treatment of patients with relapsed or refractory low-grade or follicular, CD20⁺ B cell non-Hodgkin's lymphoma. Combination therapy including the TNF-α mutein of the present invention and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, DPC-4, NF-1, NF-2, RB, p53, WT1, BRCA1 and BRCA2. The TNF-α mutein of the present invention may be used in combination with a therapy delivering the tumor suppressor in vivo (e.g., via gene therapy) to treat various forms of cancer.

In a particular embodiment, the TNF-α mutein of the present invention is combined with melphalan for treating various forms of cancer, preferably melanoma and primary limb sarcomas. The TNF-α mutein and melphalan may be further combined with interferon-γ (IFN-γ) and/or hyperthermia to treat cancer.

In another particular embodiment, the TNF-α mutein of the present invention is combined with adriamycin for treating various forms of cancer, preferably malignant lymphoma. The TNF-α mutein and adriamycin may be further combined with bleomycin, vincristine, and/or dimethyl-triazeno-imidazole carboxamide to treat cancer.

In yet another particular embodiment, the TNF-α mutein of the present invention is combined with RITUXAN® (Rituximab) for treating various forms of cancer, preferably malignant lymphoma, and more preferably non-Hodgkin's lymphomas. The TNF-α mutein and Rituximab may be further combined with other anti-neoplastic agents to treat cancer.

In yet another particular embodiment, the TNF-α mutein of the present invention is combined with cyclophosphamide for treating various forms of cancer, preferably malignant lymphoma. The TNF-α mutein and cyclophosphamide may be further combined with adriamycin, bleomycin, vincristine, and/or prednisone to treat cancer.

In yet another particular embodiment, the TNF-α mutein of the present invention is combined with mitomycin-C for treating various forms of cancer, preferably non-small cell lung cancer. The TNF-α mutein and mitomycin-C may be further combined with Vindesine (Desacetyl vinblastine amide), and/or cisplatin to treat cancer.

In yet another particular embodiment, the TNF-α mutein of the present invention is combined with vinorelbine for treating various forms of cancer, preferably non-small cell lung cancer. The TNF-α mutein and vinorelbine may be further combined with cisplatin to treat cancer.

In yet another particular embodiment, the TNF-α mutein of the present invention is combined with taxanes (e.g., paclitaxel and docetaxel) or their derivatives and analogs for treating various forms of cancer, preferably ovarian and breast cancer. The TNF-α mutein and taxane may be further combined with other anti-neoplastic agents to treat cancer.

In yet another particular embodiment, the TNF-α mutein of the present invention is combined with imatinib mesylate (or GLEEVAC®) treating various forms of cancer, preferably leukemia, gastrointestinal stromal tumor and small cell lung cancer (SCLC). The TNF-α mutein and imatinib mesylate may be further combined with other anti-neoplastic agents to treat cancer.

3. Indications for Treatment with TNF-α Mutein or its Combination with Antineoplastic Agents

Preferable indications that may be treated using the compositions of the present invention include those involving undesirable or uncontrolled cell proliferation. Such indications include benign tumors, various types of cancers such as primary tumors and tumor metastasis, hematologic disorders (e.g. leukemia, myelodysplastic syndrome and sickle cell anemia), restenosis (e.g. coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

In a particular embodiment, the TNF-α mutein of the present invention itself or in combination with other anti-neoplastic agent(s) is used to treat lung cancer, preferably non-small cell lung cancer. Lung cancer is the leading cause of cancer mortality in both men and women in the United States, and is the third most common cancer in the U.S. behind prostate and breast cancer. More Americans die from lung cancer than from breast cancer, prostate cancer, and colorectal cancer combined because of the low 14% cure rate. Parker et al. (1997) Cancer J. Clin. 47:5-27.

Four major cell types make up 95% of all primary lung neoplasma: small cell lung cancer, squamous (epidermoid) carcinomas, adenocarcinomas (including bronchoalveolar) and large-cell (undifferentiated) carcinoma. The latter three cell types are often lumped together and referred to as non-small cell lung cancer (NSCLC).

Squamous cell carcinomas, at one time, was the most frequent of all lung cancers. Squamous cell carcinomas arises most frequently in proximal segmental bronchi and is preceded by squamous metaplasia. With further growth, squamous cancers invades the basement membrane and extend into the bronchial lumen, producing obstruction with resultant atelectasis or pneumonia.

Adenocarcinoma has become the most frequent lung cancer histology in North America, accounting for approximately 35-40% of all cases of lung cancer. Most of these tumors are peripheral in origin, arising from alveolar surface epithelium or bronchial mucosal glands. They also can arise from peripheral scar tumors. Adenocarcinoma appears to have a worst prognosis for operable stages than squamous cell carcinoma because of its propensity for early metastases.

Large cell carcinoma is the least common of all NSCLC tumors, accounting for approximately 15% of all lung cancers. Most are located peripherally and are similar to adenocarcinomas in prognosis.

Small cell lung cancers have both biologic and clinical differences from NSCLC tumors. Biologically, SCLCs have nuroendocrine features. These features lead to frequent endocrine and neurologic paraneoplastic syndromes. SCLC also have more rapid growth and a greater propensity for early metastatic spread. SCLC has the most aggressive clinical course of any type of pulmonary tumor, with median survival from diagnosis of only 2 to 4 months without treatment.

In another embodiment, the TNF-α mutein of the present invention itself or in combination with other anti-neoplastic agent(s) is used to treat malignant lymphoma including both Hodgkin's disease and non-Hodgkin's lymphomas. Pathologically, Hodgkin's disease is characterized by the presence of large abnormal cells with prominent nucleoli (Reed-Sternberb cells or mononuclear variants). However, most of the tumor cells is composed of a mixture of normal-appearing inflammatory cells. Hodgkin's diseases can be divided into several histologic subtypes. The widely endorsed Rye classification specifies four categories: lymphocyte predominant, nodular sclerosis, mixed cellularity, and lymphocytes depleted. Accurate staging is crucial in planning the treatment of patients with Hodgkin's disease so that specific treatment can be tailored to the extent and location of disease.

The non-Hodgkin's lymphomas are a diverse group of neoplasms that have a common origin in the lymphoreticular cells. They usually arise or are present in the lymphoid tissues such as the lymph nodes, spleen, bone marrow, or Waldeyer's ring, but they can arise in almost any tissue. Rosenberg SA (1993) “Non-Hodgkin's lymphomas”, In Medical Oncology, second edition (Calabresi and Schein, eds.), San Francisco: McGraw-Hill, Inc., 417-33. The lymphomas include a spectrum of diseases ranging from among the most rapidly progressive and fatal human neoplasms to among the most indolent and well-tolerated, capable of prolonged stability and in some cases spontaneous regression. In the United States, approximately 35,000 new patients develop one of these neoplasms per year; about half will die of their disease.

Because of the diversity of non-Hodgkin's lymphomas, an attempt was made to develop uniform pathologic and classification systems. Based on the correlation between the pathologic subtype and survival, the non-Hodgkin's lymphomas were classified into three main grades—low, intermediate, and high—with untreated survivals longest in the low-grades and shortest in the high grades. CLL with adenopathy is classified as a low grade NHL. The median survival time for patients with low-grade lymphomas is 6.2 years from initial diagnosis. Transformation to a more aggressive cell type occurs in about 30% of patients.

The prognosis of relapsed non-Hodgkin's lymphoma (NHL) of any grade is poor. Even in patients achieving a complete response (CR) to initial induction therapy, a “poor-risk” subgroup can be defined whose chance of relapse is high and who thus have a poor overall prognosis.

In yet another embodiment, the TNF-α mutein of the present invention itself or in combination with other anti-neoplastic agent(s) is used to treat melanoma including malignant melanoma, melanoma skin cancer, and cutaneous melanoma, preferably malignant melanoma.

The most frequent human neoplasma are those of the skin. The nonmelanoma neoplasms ares chiefly squamous and basal cell carcinomas that evolve slowly, generally permitting early recognition and cure by local measures. Melanoma is a less common but more aggressive cutaneous neoplasma that has a considerable lethal potential following progression to involve regional and distant sites. The incidents of melanoma have risen more rapidly than other solid tumor; and melanoma is much more likely than basal or squamous cell cancer to metastasize (spread) to other parts of the body.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chormosomal breakage, such as Bloom's syndrome, Fanconi's anemia, Li-Fraumeni kindreds, ataxia-telangiectasia, and X-linked agammaglobulinemia.

Acute promyclocytic leukemia (APML) represents a distinct subgroup of AML. This subtype is characterized by promyelocytic blasts containing the 15;17 chromosomal translocation. This translocation leads to the generation of the fusion transcript comprised of the retinoic acid receptor and a sequence PML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient. Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanim of various angiogenic factors, including elements of the inflammatory response, such as leukocytes, platelets, cytokines, and eicosanoids, or unidentified plasma constituents have yet to be revealed.

In another embodiment of the present invention, a method is provided for treating diseases associated with undesired or abnormal angiogenesis. The method comprises administering to a patient suffering from undesired or abnormal angiogenesis a composition comprising the TNF-α mutein alone or in conjunction with an anti-angiogenesis agent.

The particular dosage of these agents required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the composition of the present invention may be used to treat a variety of diseases associated with undesirable angiogenesis such as retinal/choroidal neuvascularization and corneal neovascularization. Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

In yet another embodiment of the present invention, a method is provided for treating chronic inflammatory diseases associated with abnormal angiogenesis. The method comprises administering the TNF-α mutein of the invention in a therapeutically effective amount to a patient suffering from a chronic inflammatory disease associated with abnormal angiogenesis. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the composition of the present invention may prevent the formation of the granulosmas, thereby alleviating the disease. Examples of chronic inflammatory disease include, but are not limited to, inflammatory bowel diseases such as Crohn's disease and ulcerative colitis, psoriasis, sarcoidois, and rhematoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the composition of the present invention should inhibit the formation of the sprouts and prevent the formation of granulomas. The inflammatory bowel diseases also exhibit extra intestinal manifectations, such as skin lesions. Such lesions are characterized by inflammation and angiogenesis and can occur at many sites other the gastrointestinal tract. Inhibition of angiogenesis by the composition of the present invention should reduce the influx of inflammatory cells and prevent the lesion formation.

Sarcoidois, another chronic inflammatory disease, is characterized as a multisystem granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the composition of the present invention to inhibit angionesis, such granulomas formation can be inhibited. Psoriasis, also a chronic and recurrent inflammatory disease, is characterized by papules and plaques of various sizes. Treatment using the composition of the present invention should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the composition of the present invention alone or in conjunction with other anti-RA agents should prevent the formation of new blood vessels necessary to maintain the chronic inflammation and provide the RA patient relief from the symptoms.

According to this embodiment, the TNF-α mutein of the present invention may be used to treat a variety of diseases associated with uncontrolled angiogenesis such as retinal/choroidal neuvascularization and corneal neovascularization. Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration,, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

The TNF-α muteins of the present invention may also be used in conjunction with other anti-angiogenesis agents to inhibit undesirable and uncontrolled angiogenesis. Examples of anti-angiogenesis agents include, but are not limited to, retinoid acid and derivatives thereof, 2-methoxyestradiol, ANGIOSTATIN™ protein, ENDOSTATIN™ protein,suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,1-3,4-dehydroproline, thiaproline], α, α-dipyridyl, .beta.-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, alpha.2-antiplasmin, bisantrene, lobenzarit disodium, n-(2-carboxyphenyl-4-chloroanthronilic acid disodium or “CCA”, thalidomide; angostatic steroid, cargboxynaminolmidazole; metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

4. Routes of Administration and Dosing Regimen

A wide variety of delivery methods and formulations for different delivery methods may be used in the therapies of the present invention.

The TNF-α mutein of the present invention may be administered as compositions that comprise the TNF-α mutein. Such compositions may include, in addition to the TNF-α mutein, conventional pharmaceutical excipients, and other conventional, pharmaceutically inactive agents. Additionally, the compositions may include other active agents in addition to the TNF-α mutein. These additional active agents may include another TNF-α mutein according to the invention, or one or more other pharmaceutically active agents. In preferable embodiments, the compositions will contain the TNF-α mutein in an amount effective to treat an indication of interest.

The TNF-α mutein according to the invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The TNF-α mutein according to the invention may also be administered or co-administered in slow release dosage forms.

The TNF-α mutein may be administered by a variety of routes, and may be administered or co-administered in any conventional dosage form. Co-administration in the context of this invention is defined to mean the administration of more than one therapeutic in the course of a coordinated treatment to achieve an improved clinical outcome. Such co-administration may also be coextensive, that is, occurring during overlapping periods of time. For example, the TNF-α mutein of the present invention may be administered to a patient before, concomitantly, or after another antineoplastic agent is administered.

Amounts of the inventive combination of therapeutic agents can vary, according to determinations made by one of skill, but preferably are in amounts effective to create a cytotoxic or cytostatic effect at the desired site. Preferably, these total amounts are less than the total amount adding the maximum tolerated dose for each of the TNF-α mutein and the other antineoplastic agent, and more preferably less than the total amount added for individual administration of each of these antineoplastic agents.

For the slow-release dosage form, appropriate release times can vary, but preferably should last from about 1 hour to about 6 months, most preferably from about 1 week to about 4 weeks. Formulations including the inventive combination of therapeutic agents and/or composition can vary, as determinable by one of skill, according to the particular situation, and as generally taught herein.

Also according to the present invention, before the treatment with the TNF-α mutein, the patient may be treated with various anticancer agents described above. Owing to the sensitizing effects of the chemotherapy on the cells to apoptosis, the dosage of the TNF-α mutein used for the treatment may be lower than that used in a monotherapy of the TNF-α mutein. Thus, a better clinical outcome may be achieved with reduced side effects of the TNF-α mutein.

The TNF-α mutein according the invention may be used in the form of kits. The arrangement and construction of such kits is conventionally known to one of skill in the art. Such kits may include containers for containing the TNF-α mutein, and/or other apparatus for administering the TNF-α mutein.

The kit may optionally further include instructions. The instructions may describe how the TNF-α mutein, if supplied in a powder form, should be reconstituted with infusion fluid to form a pharmaceutical formulation. The instructions may also describe how to administer the resulting pharmaceutical formulation to a patient. It is noted that the instructions may optionally describe the administration methods according to the present invention.

The pharmaceutical formulations provided in vessels or kits may be in a form that is suitable for direct administration or may be in a concentrated form that requires dilution relative to what is administered to the patient. For example, pharmaceutical formulations, described in this invention, may be in a form that is suitable for direct administration via infusion or injection.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

### EXAMPLE

1. Expression and Purification of rhTNFm1

Recombinant human TNF-α mutant 1 (rhTNFm1, FIG. 1) was expressed in E. coli and purified by using techniques known in the art. Briefly, E. coli strain HMS174 containing a plasmid encoding rhTNFm1 was inoculated in LB (Amp) and cultured at 30° C. until OD₆₀₀ reached about 0.4. The LB culture was transferred to fermentation containers containing TH fermentation medium at 1% concentration, and cultured at cultured at 30° C. until OD₆₀₀ reached about 0.4. The pH value was maintained at 6.8-7.0 and the concentration of oxygen in the solution at 30% during the fermentation.

After the fermentation, bacterial cells were collected by centrifugation and lysed ultrasonically in cold TSE buffer (0.1 M Tris, 0.2 M NaCI, and 0.05 M EDTA at pH 7.2). The lysate was centrifuged; and the supernatant was collected. Solid ammonium sulphate was added to the supernatant to precipitate the protein by salt gradient. The protein precipitated was dissolved in 19 mM Tris.HCl and dialyzed. rhTNFm1 in the solution was purified by ion exchange columnography. Albumin was added to the purified rhTNFm1 to reach a final concentration of 1%. After the bioactivity or potency of rhTNFm1 was determined by using the international standard method for hTNF-α described in Meager and Das (1994) J. Immuno. Methods 170:1-13, the purified rhTNFm1 was packaged in ampoules at ≧500,000 international units/ampoule.

2. Clinical Treatment of Malignancy using rhTNFm1

Recombinant protein rhTNFm1 prepared above was used a therapeutic protein to treat various malignancy in the clinic, including malignant lymphoma, lung cancer, melanoma, breast cancer, liver cancer, kidney cancer, colon cancer, pleural effusion and ascites.

1) Selection of Patients

A total of 310 cancer patients have been evaluated in the clinical trial, among which 169 patients had malignant pleural effusion or ascites, and 141 patients had tumors including malignant lymphoma, lung cancer, melanoma, breast cancer, liver cancer, kidney cancer and colon cancer.

Patients were selected based on the following criteria: i) measurable, objective tumor; ii) Karnofsky performance status (KPS)≧60; iii) age 18-75; iv) major organs in basically normal condition; v) no chemotherapy, radiotherapy and biologic therapy within the last month; vi) predicted ≧3 mon. survival; and vii) with patient's informed consent signed.

Patients were excluded based on the following criteria: i) with history of allergic reactions to TNF or its derivatives; ii) pregnant or lactating women; iii) major organs with obvious damages or malfunction; iv) in ≧38° C. fever; v) with obvious tendency to haemorrhage; vi) with history of hypo- or hyper-tension; vii) with serious albuminemia; and viii) with strong tendency of disobedience or resistance.

Patients were removed from the evaluation if any of the following conditions apply: i) treatment was not completed according to the protocol; ii) incomplete records; and iii) treatment using cancer therapies not in the protocol. However, patients who withdrew due to adverse reactions to the therapy were still included in the evaluation.

The results of the treatment of tumors were evaluated based the following criteria: i) complete response (CR)—complete disappearance of observable pathological changes for at least 4 weeks; ii) partial response (PR)—reduction by ≧50% in tumor size measured by the product of the maximum circular and vertical dimensions for at least 4 weeks; iii) minimum response (MR)—reduction by >25% but <50% in tumor size measured by the product of the maximum circular and vertical dimensions for at least 4 weeks; iv) stabilized (SD)—reduction by ≦25% but without increase by >25% in tumor size measured by the product of the maximum circular and vertical dimensions for at least 4 weeks; and progression (PD): increase by >25% in tumor size in one or more sites.

The results of the treatment of malignant pleural effusion and ascites were evaluated based on a modified Millar standard (Millar J W, Hunfer A M, Home N W. (1980) “Inerapleural immunotherapy with CP”, in Recurrent Malignant Pleural Effusions. Thorax 35:856): i) complete response (CR)—complete disappearance of pleural effusion or ascites for at least 1 month; ii) partial response (PR)—reduction by at least 1 grade for at least 1 month; iii) minimum response (MR)—reduction in the levels of pleural effusion or ascites within the same grade for at least 1 month; iv) stabilized (SD)—levels of pleural effusion or ascites maintained within the same grade for at least 1 month; and progressed (PD): increase in the levels of pleural effusion or ascites compared with those before the treatment.

2) Monotherapy of rhTNFm1

Patients with the following malignancy: malignant lymphoma, lung cancer, melanoma, breast cancer, liver cancer, kidney cancer, colon cancer, pleural effusion and ascites were treated with a monotherapy of the recombinant protein rhTNFm1 described above.

Patients were treated with rhTNFm1 at 600,000-900,000 unit/m² (about 1-50 μg/m² depending on the specific bioactivity of a particular lot) per day, 5 times a week (day 3-7) via i.v. injection. Patients rested on day 1 and 2. The treatment cycle was 3-4 weeks. The results from the treatment of malignant tumors are summarized in Table I.

| | | | | | | | Table I | | |
|---|---|---|---|---|---|---|---|---|---|
| | | | | | | | Response Rate (%) | | |
| | Patient | Efficacy | | | | | | | CR + |
| Indication | Number | CR | PR | MR | SD | PD | CR | PR | PR |
| Lung Cancer | 20 | 0 | 1 | 2 | 13 | 4 | 0.00 | 5.00 | 5.00 |
| Liver Cancer | 12 | 0 | 0 | 0 | 10 | 2 | 0.00 | 0.00 | 0.00 |
| Colon Cancer | 7 | 0 | 0 | 0 | 3 | 4 | 0.00 | 0.00 | 0.00 |
| Breast Cancer | 10 | 0 | 0 | 0 | 6 | 4 | 0.00 | 0.00 | 0.00 |
| Kidney Cancer | 3 | 0 | 0 | 1 | 1 | 1 | 0.00 | 0.00 | 0.00 |
| Malignant | 71 | 2 | 18 | 8 | 32 | 11 | 2.82 | 25.35 | 28.17 |
| Lymphoma | | | | | | | | | |
| Malignant | 18 | 0 | 2 | 3 | 10 | 3 | 0.00 | 11.11 | 11.11 |
| melanoma | | | | | | | | | |
| Total* | 141 | 2 | 21 | 14 | 75 | 29 | 1.42 | 14.89 | 16.31 |

It is noted that Table I includes the patients who withdrew due to ineffectiveness of the treatment, including 3 with malignant lymphoma, 1 with liver cancer, and 1 with malignant melanoma.

Table II summaries results of the treatment of patients with malignant lymphoma, classified according to Hodgkin's disease and non-Hodgkin's lymphoma. Within each of category of lymphoma, patients were further classified into two groups: chemo-naïve and chemo-refractory. The chemo-naive patients were patients who had never been treated with any anti-cancer agent previously whereas chemo-refractory patients were ones who failed treatment with one or more anti-cancer agent. As shown in Table II, patients with non-Hodgkin's lymphoma were responsive to the treatment with rhTNFm1 alone. Noticeably, two chemo-refractory patients had complete response to the treatment with rhTNFm1 alone.

TABLE II

| | | | Patient | Efficacy | | | | | Response Rate (%) | | |
|---|---|---|---|---|---|---|---|---|---|---|---|
| Lymphoma | Treatment | | # | CR | PR | MR | SD | PD | CR | PR | CR + PR |
| Hodgkin's | Chemo- | Grade I & | 0 | 0 | 0 | 0 | 0 | 0 | 0 | 0 | 0 |
| | naïve | II | | | | | | | | | |
| | Patients | III & IV | 0 | 0 | 0 | 0 | 0 | 0 | 0 | 0 | 0 |
| | | Total | 0 | 0 | 0 | 0 | 0 | 0 | 0 | 0 | 0 |
| | Chemo-refractory | | 6 | 0 | 0 | 0 | 5 | 1 | 0 | 0 | 0 |
| | Patients | | | | | | | | | | |
| Non- | Chemo- | Grade I & | 4 | 0 | 3 | 1 | 0 | 0 | 0 | 75 | 75 |
| Hodgkin's | naïve | II | | | | | | | | | |
| | Patients | III & IV | 5 | 0 | 3 | 0 | 2 | 0 | 0 | 60 | 60 |
| | | Total | 9 | 0 | 6 | 1 | 2 | 0 | 0 | 66.67 | 66.67 |

| | | | | | | | | | |
|---|---|---|---|---|---|---|---|---|---|
| Chemo-refractory | 56 | 2 | 12 | 7 | 25 | 10 | 3.57 | 21.43 | 25 |
| Patients | | | | | | | | | |

| | | | | | | | | | |
|---|---|---|---|---|---|---|---|---|---|
| Total | 71 | 2 | 18 | 8 | 32 | 11 | 2.82 | 25.35 | 28.17 |

Table III summarizes results of the treatment of patients with malignant pleural effusion or ascites. As shown in Table III, these patients were responsive to the treatment with rhTNFm1 alone.

TABLE III

| | | | | | | | Response Rate (%) | | |
|---|---|---|---|---|---|---|---|---|---|
| | Patient | Efficacy | | | | | | | CR + |
| Indication | # | CR | PR | MR | SD | PD | CR | PR | PR |
| pleural | 141 | 14 | 91 | 9 | 26 | 1 | 9.93 | 64.54 | 74.47 |
| effusion | | | | | | | | | |
| Ascites | 28 | 1 | 11 | 3 | 12 | 1 | 3.57 | 39.29 | 42.86 |
| Total | 169 | 15 | 102 | 12 | 38 | 2 | 8.88 | 60.36 | 69.23 |

It is noted that Table III includes the patients who withdrew due to ineffectiveness of the treatment, including 1 with malignant pleural effusion and 1 with malignant ascites. For patients with malignant ascites, rhTNFm1 was reconstituted in 30-50 ml normal saline and administered intraperitoneally (i.p.) at 2,000,000-3,000,000 units each time, 1-2 times a week, with 2-3 weeks as one treatment cycle. Optionally, rhTNFm1 was administered to the patients by alternating between i.p. injection and i.v. injection (at 600,000-900,000 unit/m² per day continuing for 28 days as one treatment cycle). On the day when rhTNFm1 was administered via i.p. injection, administration of rhTNFm1 via i.v. injection was stopped.

Based on the results described above, monotherapy of rhTNFm1 was particularly efficacious in the treatment of non-Hodgkin's lymphoma, lung cancer, malignant melanoma, and malignant pleural effusion and ascites. Notably, the response rate was especially high (above 70%) in the treatment of patients with malignant pleural effusion with rhTNFm1 alone.

During the treatment with rhTNFm1 alone, adverse effects observed were quite mild, mainly fever and chills. About 38% of the patients treated had fever that was below the 2^{nd} degree; and there were only two patients with 3^{rd} degree fever. Only 23% of the patients treated had chills, among them 12% slight chills and 11% mild chills. The rest of the patients had neither fever nor chills.

Chills often occurred within 30 min after the administration of rhTNFm1, sometimes after 1 hr post treatment, and usually lasted no more than half an hour. Fever often occurred within 1-2 hr post administration of rhTNFm1 and usually lasted no more than half an hour, which was alleviated by using mezolin suppository prophylactically and/or post treatment with rhTNFm1. Occurrence of fever and chills gradually declined with the days of treatment with rhTNFm1: about 24% and 17% on day 1, about 10% and 5% on day 7, and about 5% and 2% on day 21, respectively. These results show that compared to wild type TNF-α, the therapy using rhTNFm1 according to the present invention caused much fewer and weaker adverse effects on the patients. In addition, the KPS of the patients in the test groups was significantly improved compared to that prior to the treatment (P=0.013), particularly in the treatment of malignant pleural effusion.

3) Combination therapy of rhTNFm1 and other anti-neoplastic agents

Patients with the malignant lymphoma and non-small cell lung cancer were treated with a combination of rhTNFm1 and chemotherapy.

a) Malignant lymphoma

Patients with malignant lymphoma were divided according to Hodgkin's disease (HD) and non-Hodgkin's lymphoma (NHL).

HD patients were treated with a combination of rhTNFm1, adriamycin, bleomycin, vincristine and dimethyl-triazeno-imidazole carboxamide. Specifically, adriamycin was administered to the patients at 25 mg/m² (E-ADM at 40 mg/m²) via i.v. injection on day 1 and day 15; bleomycin at 10 mg/m² via i.v. injection on day 1 and day 15; vincristine at 2 mg via i.v. injection on day 1 and day 15; and dimethyl-triazeno-imidazole carboxamide at 375 mg/m² via i.v. infusion on day 1 and day 15. rhTNFm1 was administered to the patients at 600,000-900,000 unit/m² (about 1-50 μg/m² depending on the specific bioactivity of a particular lot) per day, 5 times a week (day 3-7). Patients rested on day 1 and 2. The treatment cycle was 28 days; and patients were evaluated for efficacy after 2 cycles of treatment.

NHL patients were treated with a combination of rhTNFm1, cyclophosphamide, vincristine, adriamycin, bleomycin, and prednisone. Specifically, cyclophosphamide was administered to the patients at 650 mg/m² via i.v. injection on day 1 and day 8; vincristine at 1.4 mg/m² via i.v. injection on day 1 and day 8; adriamycin at 25 mg/m² (or E-ADM at 40 mg/m²) via i.v. perfusion on day 1 and day 8; bleomycin at 10 mg/m² via i.m. injection on day 15 and day 22; and prednisone at 100 mg/m² via p.o. on day 15-28. rhTNFm1 was administered to the patients at 600,000-900,000 unit/m² (about 1-50 μg/m² depending on the specific bioactivity of a particular lot) per day, 5 times a week (day 3-7). Patients rested on day 1 and 2. The treatment cycle was 28 days; and patients were evaluated for efficacy after 2 cycles of treatment.

The results from the treatment of malignant lymphoma with the combination therapy are summarized in Table IV. Comparison (Z value) of the test group (treated with combination of rhTNFm1 and chemotherapy) and the control group (treated with chemotherapy alone) was calculated using Wilcoxon method.

TABLE IV

| | | | Patient | Efficacy | | | | | Response Rate (%) | | | Z | P |
|---|---|---|---|---|---|---|---|---|---|---|---|---|---|
| Indication | | Group | # | CR | PR | MR | SD | PD | CR | PR | CR + PR | Value | Value |
| HD | Chemo- | Test | 3 | 1 | 2 | 0 | 0 | 0 | 33.33 | 66.67 | 100.00 | 0.2 | 0.814 |
| | Naive | Control | 3 | 1 | 1 | 1 | 0 | 0 | 33.33 | 33.33 | 66.67 | — | — |
| | Chemo- | Test | 5 | 3 | 1 | 0 | 1 | 0 | 60.00 | 20.00 | 80.00 | 0.9 | 0.347 |
| | Ref. | Control | 3 | 0 | 2 | 1 | 0 | 0 | 0.00 | 66.67 | 66.67 | — | — |
| NHL | Chemo- | Test | 27 | 7 | 18 | 1 | 0 | 1 | 25.93 | 66.67 | 92.59 | 0.4 | 0.668 |
| | Naive | Control | 15 | 5 | 6 | 0 | 4 | 0 | 33.33 | 40.00 | 73.33 | — | — |
| | Chemo- | Test | 16 | 4 | 8 | 1 | 3 | 0 | 25.00 | 50.00 | 75.00 | 2.4 | 0.018 |
| | Ref. | Control | 10 | 0 | 3 | 2 | 4 | 1 | 0.00 | 30.00 | 30.00 | — | — |

| | | | | | | | | | | | | |
|---|---|---|---|---|---|---|---|---|---|---|---|---|
| Malignant | Test | 51 | 14 | 30 | 2 | 4 | 1 | 27.45 | 58.82 | 86.27 | 2.4 | 0.015 |
| Lymphoma | Control | 31 | 6 | 11 | 3 | 10 | 1 | 19.35 | 35.48 | 54.84 | — | — |

These results indicate that in the treatment of malignant lymphoma in general the response rate of rhTNFm1+chemo combination therapy (˜86%) is higher than that of chemotherapy alone (˜55%); and the difference is statistically significant (P=0.015). In particular, NHL patients treated with rhTNFm1+chemo combination therapy had a much higher response rate (˜75%) than that of chemotherapy alone (˜30%); and the difference is statistically significant (P=0.018). In addition, the KPS of the patients in the test group was significantly improved compared to that prior to the treatment (P=0.004). In contrast, there was no significant improvement in the KPS of the patients in the control group compared to that prior to the treatment.

b) Non-small Cell Lung Cancer (NSCLC)

NSCLC patients were treated with a combination of rhTNFm1, mitomycin-C, Vindesine (Desacetyl vinblastine amide), and cisplatin. Specifically, , mitomycin-C was administered to the patients at 6 mg/m² via i.v. perfusion on day 1 and day 8; Vindesine at 3 mg/m² via i.v. perfusion on day 1 and day 8; and cisplatin at 50 mg/m² via i.v. infusion on day 3 and day 4. rhTNFm1 was administered to the patients at 600,000-900,000 unit/m² (about 1-50 μg/m² depending on the specific bioactivity of a particular lot) per day, 5 times a week (day 3-7). Patients rested on day 1 and 2. The treatment cycle was 21 days; and patients were evaluated for efficacy after 2 cycles of treatment.

Alternatively, NSCLC patients were treated with a combination of rhTNFm1, Vinorelbine (i.e., Navelbin), and cisplatin. Specifically, Vinorelbine was administered to the patients at 25 mg/m² via i.v. perfusion on day 1 and day 8; and cisplatin at 80-100 mg/m² via i.v. infusion on day 1. rhTNFm1 was administered to the patients at 600,000-900,000 unit/m² (about 1-50 μg/m² depending on the specific bioactivity of a particular lot) per day, 5 times a week (day 3-7). Patients rested on day 1 and 2. The treatment cycle was 21 days; and patients were evaluated for efficacy after 2 cycles of treatment.

The results from the treatment of NSCLC with the combination therapy are summarized in Table V. Comparison (Z value) of the test group (treated with combination of rhTNFm1 and chemotherapy) and the control group (treated with chemotherapy alone) was calculated using the Wilcoxon method.

TABLE V

| | | Patient | Efficacy | | | | | Response Rate (%) | | | Z | P |
|---|---|---|---|---|---|---|---|---|---|---|---|---|
| Indication | Group | # | CR | PR | MR | SD | PD | CR | PR | CR + PR | value | Value |
| NSCLC | Test | 44 | 0 | 14 | 6 | 21 | 3 | 0.00 | 31.82 | 31.82 | 1.9 | 0.052 |
| | Control | 25 | 0 | 4 | 1 | 17 | 3 | 0.00 | 16.00 | 16.00 | — | — |

These results indicate that in the treatment of NSCLC the response rate of rhTNFm1+chemo combination therapy (˜32%) is higher than that of chemotherapy alone (˜16%); and the difference is statistically significant (P=0.015). In addition, quality of life for the patients in the test group was significantly improved compared to that prior to the treatment (P=0.001). In contrast, there was no significant improvement in quality of time for the patients in the control group compared to that prior to the treatment (P=0.668).

What is claimed is:

1. A method for treating a patient having a disease associated with abnormal proliferation of cells, comprising:
administering to the patient a mutein of human tumor necrosis factor-alpha (hTNF-α), the hTNF-α mutein being defined based on the amino acid sequence of hTNF-α (SEQ ID NO: 1) as having a deletion of amino acid residues at positions 1-7, wherein the amino acid residue positions are numbered relative to the N-terminus of hTNF-α.

2. The method of claim 1, wherein the hTNF-α mutein further has a substitution of amino acid residue either at position 156 or 157 with a residue selected from the group consisting of Gln, Ser, Thr, Tyr, and Asn.

3. The method of claim 1, wherein the hTNF-α mutein further has a substitution of one or more of amino acid residues at positions 8-10 with Lys or Arg.

4. The method of claim 1, wherein the hTNF-α mutein has an amino acid sequence comprising SEQ ID NO: 2, 3, 4, or 5.

5. The method of claim 1, wherein the hTNF-α mutein is encoded by a DNA sequence comprising SEQ ID NO: 10.

6. The method of claim 1, wherein the disease associated with abnormal proliferation of cells is selected from the group consisting of hematological disorders, cancer, malignant pleural effusion, malignant ascite, restenosis, and inflammatory diseases.

7. The method of claim 6, wherein the hematologic disorders are selected from the group consisting of acute myeloid leukemia, acute promyelocytic leukemia, acute lymphoblastic leukemia, chronic myelogenous leukemia, the myelodysplastic syndromes, and sickle cell anemia.

8. The method of claim 6, wherein the cancer is selected from the group consisting of breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas.

9. The method of claim 6, wherein the cancer is lung cancer.

10. The method of claim 9, wherein the lung cancer is non-small cell lung cancer.

11. The method of claim 6, wherein the cancer is melanoma or skin cancer.

12. The method of claim 11, wherein the melanoma is malignant or metastatic melanoma.

13. The method of claim 6, wherein the cancer is lymphoma, either Hodgkin's disease or non-Hodgkin's lymphomas.

14. The method of claim 6, wherein the cancer is non-Hodgkin's lymphomas.

15. The method of claim 1, wherein the disease associated with abnormal proliferation of cells is malignant pleural effusion.

16. The method of claim 1, wherein the disease associated with abnormal proliferation of cells is malignant ascites.

17. The method of claim 1, wherein the hTNF-α mutein is administered to the patient orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally.

18. The method of claim 1, wherein the hTNF-α mutein is administered the patient in a slow release dosage form.

19. The method of claim 1, wherein the hTNF-α mutein is administered intravenously.

20. The method of claim 1, wherein the hTNF-α mutein is administered intratumorally, intraperitoneally, by isolated limb perfusion, by isolated lung perfusion, by isolated liver perfusion, or by intravesical or intra-arterial infusion.

21. The method of claim 1, wherein the hTNF-α mutein is administered to the patient via intravenous injection per day for 3-5 days per week at a dose of 0.1-100 μg/m².

22. The method of claim 1, wherein the hTNF-α mutein is administered to the patient via intravenous injection per day for 3-5 days per week at a dose of 0.5-50 μg/m².

23. The method of claim 1, wherein the hTNF-α mutein is administered to the patient via intravenous injection per day for 3-5 days per week at a dose of 1-20 μg/m².

24. The method of claim 1, wherein the hTNF-α mutein is administered to the patient via intravenous injection per day for 3-5 days per week at a dose of 100,000-1,000,000 unit/m².

25. The method of claim 1, wherein the hTNF-α mutein is administered to the patient via intravenous injection per day for 3-5 days per week at a dose of 200,000-800,000 unit/m².

26. The method of claim 1, wherein the hTNF-α mutein is administered to the patient via intravenous injection per day for 3-5 days per week at a dose of 400,000-800,000 unit/m².

27. The method of claim 1, wherein the hTNF-α mutein is administered to the patient via intraperitoneal injection per day for 1-2 days per week at a dose of 500,000-5,000,000 units.

28. The method of claim 1, wherein the hTNF-α mutein is administered to the patient via intraperitoneal injection per day for 1-2 days per week at a dose of 1,000,000-3,000,000 units.

29. The method of claim 1, wherein the hTNF-α mutein is administered to the patient via intraperitoneal injection per day for 1-2 days per week at a dose of 2,000,000-3,000,000 units.

30. The method of claim 1, further comprising:
administering to the patient an anti-neoplastic agent other than the hTNF-α mutein itself.

31. The method of claim 30, wherein the anti-neoplastic agent is selected from the group consisting of alkylating agent, antibiotic agent, antimetabolic agent, hormonal agent, plant-derived agent, anti-angiogenesis agent and biologic agent.

32. The method of claim 31, wherein the alkylating agent is selected from the group consisting of bischloroethylamines, aziridines, alkyl alkone sulfonates, nitrosoureas, nonclassic alkylating agents and platinum compounds.

33. The method of claim 31, wherein the antibiotic agent is selected from the group consisting of doxorubicin, daunorubicin, epirubicin, idarubicin and anthracenedione, mitomycin C, bleomycin, dactinomycin, and plicatomycin.

34. The method of claim 31, wherein the antimetabolic agent is selected from the group consisting of fluorouracil, floxuridine, methotrexate, leucovorin, hydroxyurea, thioguanine, mercaptopurine, cytarabine, pentostatin, fludarabine phosphate, cladribine, asparaginase, and gemcitabine.

35. The method of claim 31, wherein the hormonal agent is selected from the group consisting of diethylstibestrol, tamoxifen, toremifene, fluoxymesterol, raloxifene, bicalutamide, nilutamide, flutamide, aminoglutethimide, tetrazole, ketoconazole, goserelin acetate, leuprolide, megestrol acetate and mifepristone.

36. The method of claim 31, wherein the plant-derived agent is selected from the group consisting of vincristine, vinblastine, vindesine, vinzolidine, vinorelbine, etoposide teniposide, camptothecin, paclitaxel and docetaxel.

37. The method of claim 31, wherein the biologic agent is selected from the group consisting of immuno-modulating proteins, monoclonal antibodies against tumor antigens, tumor suppressor genes, and cancer vaccines.

38. The method of claim 37, wherein the immuno-modulating protein is selected from the group consisting of interleukin 2, interleukin 4, interleukin 12, interferon α, interferon β, interferon γ, erythropoietin, granulocyte-CSF, granulocyte, macrophage-CSF, bacillus Calmette-Guerin, levamisole, and octreotide.

39. The method of claim 37, wherein the monoclonal antibody against tumor antigen is Trastruzumab or Rituximab.

40. The method of claim 37, wherein the tumor suppressor gene is selected from the group consisting of DPC-4, NF-1, NF-2, RB, p53, WT1, BRCA, and BRCA2.

41. The method of claim 37, wherein the cancer vaccine is selected from the group consisting of gangliosides, prostate specific antigen, α-fetoprotein, carcinoembryonic antigen, melanoma associated antigen MART-1, gp100, papillomavirus E6 fragment, papillomavirus E7 fragment, whole cells or portions/lysate of antologous tumor cells, and allogeneic tumor cell.

42. The method of claim 41, further comprising: administering to the patient an adjuvant to augment the immune response to the cancer vaccine.

43. The method of claim 42, wherein the adjuvant is selected from the group consisting of bacillus Calmette-Guerin, endotoxin lipopolysaccharides, keyhole limpet hemocyanin, interleukin-2, granulocyte-macrophage colony-stimulating factor, and cytoxan.

44. The method of claim 30, wherein the anti-neoplastic agent is melphalan, and the disease associated with abnormal proliferation of cells is melanoma or primary limb sarcomas.

45. The method of claim 44, further comprising:
administering to the patient interferon-γ and/or hyperthermia.

46. The method of claim 30, wherein the anti-neoplastic agent is adriamycin, and the disease associated with abnormal proliferation of cells is malignant lymphoma.

47. The method of claim 46, further comprising:
administering to the patient bleomycin, vincristine, and/or dimethyl-triazeno-imidazole carboxamide.

48. The method of claim 30, wherein the anti-neoplastic agent is Rituximab, and the disease associated with abnormal proliferation of cells is non-Hodgkin's lymphomas.

49. The method of claim 30, wherein the anti-neoplastic agent is cyclophosphamide, and the disease associated with abnormal proliferation of cells is malignant lymphoma.

50. The method of claim 49, further comprising:
administering to the patient adriamycin, bleomycin, vincristine, and/or prednisone.

51. The method of claim 30, wherein anti-neoplastic agent is mitomycin-C, and the disease associated with abnormal proliferation of cells is non-small cell lung cancer.

52. The method of claim 51, further comprising:
administering to the patient Vindesine and/or cisplatin.

53. The method of claim 30, wherein the anti-neoplastic agent is vinorelbine, and the disease associated with abnormal proliferation of cells is non-small cell lung cancer.

54. The method of claim 53, further comprising:
administering to the patient cisplatin.

55. The method of claim 30, wherein the anti-neoplastic agent is paclitaxel or docetaxel, and the disease associated with abnormal proliferation of cells is ovarian or breast cancer.

56. The method of claim 30, wherein the anti-neoplastic agent is imatinib mesylate (or GLEEVAC®).

57. The method of claim 56, wherein the disease associated with abnormal proliferation of cells is leukemia, gastrointestinal stromal tumor or small cell lung cancer.

58. A method for treating a patient having a disease associated with abnormal proliferation of cells, comprising:
administering to the patient a mutein of human tumor necrosis factor-alpha (hTNF-α), the hTNF-α mutein being defined based on the amino acid sequence of hTNF-α (SEQ ID NO: 1) as having a substitution of amino acid residues at positions 80, 90, and 92, wherein the amino acid residue positions are numbered relative to the N-terminus of hTNF-α.

59. The method of claim 58, wherein the hTNF-α mutein has an amino acid sequence comprising SEQ ID NO: 6 or 7.

60. The method of claim 58, wherein the disease associated with abnormal proliferation of cells is selected from the group consisting of hematological disorders, cancer, malignant pleural effusion, malignant ascite, restenosis, and inflammatory diseases.

61. A method for treating a patient having a disease associated with abnormal proliferation of cells, comprising: administering to the patient a mutein of human tumor necrosis factor-alpha (hTNF-α), the hTNF-α mutein being defined based on the amino acid sequence of hTNF-α (SEQ ID NO: 1) as having a substitution of amino acid residue at position 2, wherein the amino acid residue positions are numbered relative to the N-terminus of hTNF-α.

62. The method of claim 61, wherein the hTNF-α mutein has an amino acid sequence comprising SEQ ID NO: 8 or 9.

63. The method of claim 61, wherein the disease associated with abnormal proliferation of cells is selected from the group consisting of hematological disorders, cancer, malignant pleural effusion, malignant ascite, restenosis, and inflammatory diseases.


SEQUENCE LISTING (11 sequences)

SEQ ID NO: 1 - protein, 157 aa, Homo sapiens [sequence omitted]

SEQ ID NO: 2 - protein, 150 aa, Artificial Sequence
  rhTNFm1 without Met
RKRKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAQ

SEQ ID NO: 3 - protein, 151 aa, Artificial Sequence
  rhTNFm1 with Met
MRKRKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAQ

SEQ ID NO: 4 - protein, 144 aa, Artificial Sequence
  rhTNFm2 without Met
PSDKPVAHVVANPQAEGQLQWLNRRANALLANGVELRDNQLVVPSEGLYLIYSQVLFKGQGCPSTHVLLTHTISRIAVSYQTKVNLLSAIKSPCGAEAKPWYEPIYLGGVFQLEKGDRLSAEINRPDYLDFAESGQVYFGIIAL

SEQ ID NO: 5 - protein, 145 aa, Artificial Sequence
  rhTNFm2 with Met
MPSDKPVAHVVANPQAEGQLQWLNRRANALLANGVELRDNQLVVPSEGLYLIYSQVLFKGQGCPSTHVLLTHTISRIAVSYQTKVNLLSAIKSPCGAEAKPWYEPIYLGGVFQLEKGDRLSAEINRPDYLDFAESGQVYFGIIAL

SEQ ID NO: 6 - protein, 157 aa, Artificial Sequence
  rhTNFm3 without Met
VRSSSRTPSDKPVAHVVANPQAEGQLQWLNRRANALLANGVELRDNQLVVPSEGLYLIYSQVLFKGQGCPSTHVLLTHTSSRIAVSYQTHVVLLSAIKSPCQRETPEGAEAKPWYEPIYLGGVFQLEKGDRLSAEINRPDYLDFAESGQVYFGIIAL

SEQ ID NO: 7 - protein, 158 aa, Artificial Sequence
  rhTNFm3 with Met
MVRSSSRTPSDKPVAHVVANPQAEGQLQWLNRRANALLANGVELRDNQLVVPSEGLYLIYSQVLFKGQGCPSTHVLLTHTSSRIAVSYQTHVVLLSAIKSPCQRETPEGAEAKPWYEPIYLGGVFQLEKGDRLSAEINRPDYLDFAESGQVYFGIIAL

SEQ ID NO: 8 - protein, 157 aa, Artificial Sequence
  rhTNFm4 without Met
VKSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 9 - protein, 158 aa, Artificial Sequence
  rhTNFm4 with Met
MVKSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 10 - DNA, 453 bp, Artificial Sequence
  DNA encoding rhTNFm1 with Met
atgcgcaaacgtaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgcccag

SEQ ID NO: 11 - protein, 76 aa, Homo sapiens
MSTESMIRDVELAEEALPKKTGGPQGSRRCLFLSLFSFLIVAGATTLFCLLHFGVIGPQREESPRDLSLISPLAQA